(19) United States
(12) Patent Application Publication
(10) Pub. No.: US 2016/0183830 A1
(43) Pub. Date: Jun. 30, 2016

(54) MEDICAL DEVICES FOR MAPPING CARDIAC TISSUE

(71) Applicant: Boston Scientific Scimed Inc., Maple Grove, MN (US)

(72) Inventors: Jacob I. Laughner, St. Paul, MN (US); Shibaji Shome, Arden Hills, MN (US); Pramodsingh H. Thakur, Woodbury, MN (US); Kevin J. Stalsberg, White Bear Lake, MN (US)

(21) Appl. No.: 14/874,237

(22) Filed: Oct. 2, 2015

Related U.S. Application Data
(60) Provisional application No. 62/059,656, filed on Oct. 3, 2014.

(51) Int. Cl. A61B 5/04; A61B 5/00; A61B 5/0432; A61B 5/044; A61B 5/042

(52) U.S. Cl. CPC A61B 5/04014; A61B 5/044; A61B 5/0422; A61B 5/0432; A61B 5/7257; A61B 5/726; A61B 5/6858

(57) ABSTRACT

Medical devices and methods for making and using medical devices are disclosed. An example system for mapping the electrical activity of the heart includes a processor. The processor is capable of sensing a plurality of signals with a plurality of electrodes positioned within the heart and collecting a plurality of signals corresponding to the plurality of electrodes. Collecting the plurality of signals occurs over a time period. The processor is also capable of generating a plurality of time-frequency distributions corresponding the plurality of signals, generating a composite time-frequency distribution corresponding to the plurality of signals, generating a filter from the composite time-frequency distribution and applying the filter to the plurality of signals or to the plurality of time-frequency distributions

### CROSS-REFERENCE TO RELATED APPLICATION

This application claims priority to Provisional Application No. 62/059,656, filed Oct. 3, 2014, which is herein incorporated by reference in its entirety.

### TECHNICAL FIELD

The present disclosure pertains to medical devices, and methods for manufacturing medical devices. More particularly, the present disclosure pertains to medical devices and methods for mapping and/or ablating cardiac tissue.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example system for mapping the electrical activity of the heart includes a processor. The processor is capable of sensing a plurality of signals with a plurality of electrodes positioned within the heart and collecting a plurality of signals corresponding to the plurality of electrodes. Collecting the plurality of signals occurs over a time period. The processor is also capable of generating a plurality of time-frequency distributions corresponding the plurality of signals, generating a composite time-frequency distribution corresponding to the plurality of signals, generating a filter from the composite time-frequency distribution and applying the filter to the plurality of signals or to the plurality of time-frequency distributions.

Alternatively or in addition to any of the examples above, generating a plurality of time-frequency distributions utilizes at least one Fourier transform, Short-Time Fourier transform and/or a Wavelet transform.

Alternatively or in addition to any of the examples above generating a plurality of time-frequency distributions includes utilizing a Continuous Wavelet Transform in conjunction with a Fourier transform.

Alternatively or in addition to any of the examples above, each of the plurality of time-frequency distributions includes one or more frequency values occurring at one or more frequencies and one or more time points. Additionally, generating a composite time-frequency distribution includes determining the mode, median or mean of all the time-frequency distributions at each frequency and time point.

Alternatively or in addition to any of the examples above, generating a filter from the composite time-frequency distribution includes identifying a dominant frequency value for each time point of the composite time-frequency distribution and each dominant frequency value corresponds to a dominant frequency characteristic.

Alternatively or in addition to any of the examples above, the dominant frequency characteristic includes a maximum frequency value, a chirp, a sustained maximum frequency value, a local maximum frequency and/or a dominant frequency characteristic.

Alternatively or in addition to any of the examples above, generating a filter from the composite time-frequency distribution includes generating a binary mask.

Alternatively or in addition to any of the examples above, generating the binary mask includes a dominant frequency region defined between a maximum frequency and a minimum frequency.

Alternatively or in addition to any of the examples above, generating the binary mask includes a dominant frequency region defined between a first bound that corresponds to a percentage increase for each dominant frequency value over time and a second bound that corresponds to a percentage decrease for each dominant frequency value over time.

Alternatively or in addition to any of the examples above, generating the filter from the composite time-frequency distribution includes multiplying the binary mask with each of the plurality of time-frequency distributions.

Alternatively or in addition to any of the examples above, multiplying the binary mask values with each of the plurality of time-frequency distributions generates an alternate time-frequency distribution corresponding to each of the time-frequency distributions.

Alternatively or in addition to any of the examples above, the system may include generating a visual display and the visual display includes displaying at least one visual indicator and the visual indicator corresponds to each alternate time-frequency distribution.

Alternatively or in addition to any of the examples above, generating a visual display includes displaying at least one sinusoid corresponding to each of the alternate time-frequency distributions.

Alternatively or in addition to any of the examples above, displaying at least one sinusoid includes performing an Inverse Continuous Waveform transform on each alternate time-frequency distribution.

Alternatively or in addition to any of the examples above, the visual display includes displaying a phase map and wherein the visual indicator is a color, texture or both.

Another example system for mapping the electrical activity of the heart includes a catheter shaft, a plurality of electrodes coupled to the catheter shaft and a processor. The processor is capable of sensing a plurality of signals with a plurality of electrodes positioned within the heart and collecting a plurality of signals corresponding to the plurality of electrodes. Collecting the plurality of signals occurs over a time period and the time period includes one or more time points. The system is also capable of generating a plurality of time-frequency distributions corresponding to the plurality of signals and generating a composite time-frequency distribution corresponding to the plurality of time-frequency distributions. Further, the composite time-frequency distribution includes one or more fundamental frequency values at each time point of the time period. The processor is also capable of generating a filter from the composite time-frequency distribution and applying the filter to the plurality of time-frequency distributions.

Alternatively or in addition to any of the examples above, generating a filter from the composite time-frequency distribution includes generating a binary mask corresponding to the fundamental frequency values.

Alternatively or in addition to any of the examples above, applying the filter to the plurality of time-frequency distributions further includes multiplying the binary mask with the time-frequency distributions to generate an alternate time-frequency distribution for each electrode.

Alternatively or in addition to any of the examples above, creating a visual display includes displaying a sinusoid corresponding to the alternate time-frequency distribution for each electrode.

An example method for mapping the electrical activity of the heart includes positioning a mapping device in the heart. The mapping device is coupled to a processor and the processor is capable of sensing a plurality of signals with a plurality of electrodes positioned within the heart, collecting a plurality of signals corresponding to the plurality of electrodes. Collecting the plurality of signals occurs over a time period. The method also includes generating a plurality of time-frequency distributions corresponding the plurality of signals, generating a composite time-frequency distribution corresponding to the plurality of signals, generating a filter from the composite time-frequency distribution and applying the filter to the plurality of time-frequency distribution.

The above summary of some embodiments is not intended to describe each disclosed embodiment or every implementation of the present disclosure. The Figures, and Detailed Description, which follow, more particularly exemplify these embodiments.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

The disclosure may be more completely understood in consideration of the following detailed description in connection with the accompanying drawings, in which:

FIG. 1 is a schematic view of an example catheter system for accessing a targeted tissue region in the body for diagnostic and therapeutic purposes;

FIG. 2 is a schematic view of an example mapping catheter having a basket functional element carrying structure for use in association with the system of FIG. 1;

FIG. 3 is a schematic view of an example functional element including a plurality of mapping electrodes;

FIG. 4 is an illustration of an example electrogram signal in the time domain and a corresponding frequency representation in the frequency domain;

FIG. 5 is an illustration of an example time-frequency representation for a single electrode;

FIG. 6 is an illustration of an example two-dimensional composite time-frequency representation;

FIG. 7 is an illustration of an example time-frequency representation including a selection band;

FIG. 8 is an illustration of an example time-frequency representation including a selection band;

FIG. 9 is an illustration of an example binary mask;

FIG. 10 is an illustration of an example binary mask.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an example”, “some examples”, “other examples”, etc., indicate that the example described may include one or more particular features, structures, and/or characteristics. However, such recitations do not necessarily mean that all examples include the particular features, structures, and/or characteristics. Additionally, when particular features, structures, and/or characteristics are described in connection with one example, it should be understood that such features, structures, and/or characteristics may also be used in connection with other examples whether or not explicitly described unless clearly stated to the contrary. Also, when particular features, structures, and/or characteristics are described in connection with one example, it is implicit that other examples may include less than all of the disclosed features, structures, and/or characteristics in all combinations.

The following detailed description should be read with reference to the drawings in which similar elements in different drawings are numbered the same. The drawings, which are not necessarily to scale, depict illustrative embodiments and are not intended to limit the scope of the disclosure.

Mapping the electrophysiology of heart rhythm disorders often involves the introduction of a basket catheter (e.g. Constellation) or other mapping/sensing device having a plurality of sensors into a cardiac chamber. The sensors, for example electrodes, detect physiological signals, such as cardiac electrical activity, at sensor locations. It may be desirable to have detected cardiac electrical activity processed into electrogram signals that accurately represent cellular excitation through cardiac tissue relative to the sensor locations. A processing system may then analyze and output the signal to a display device. Further, the processing system may output the signal as processed output, such as a static or dynamic activation map. A user, such as a physician, may use the processed output to perform a diagnostic procedure.

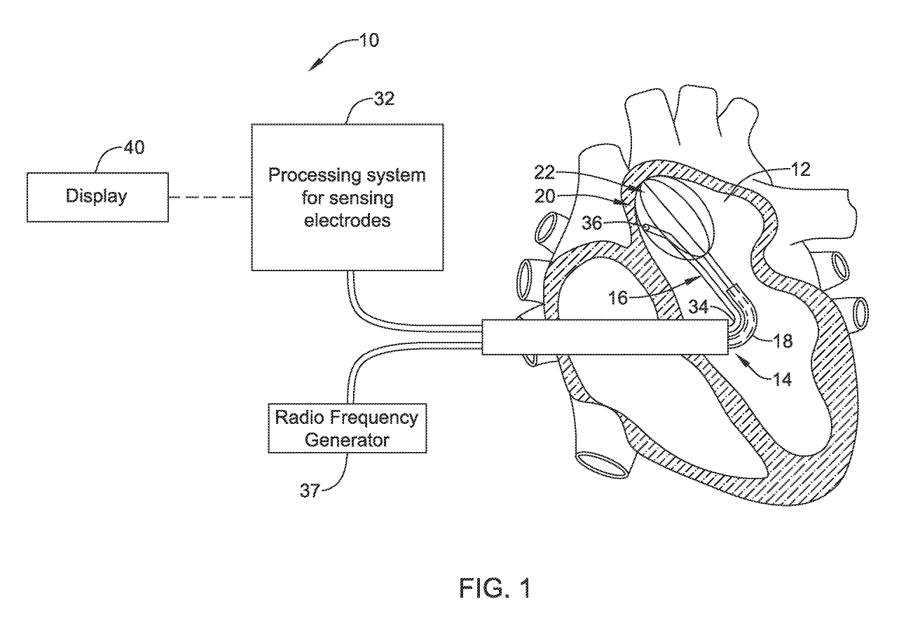

FIG. 1 is a schematic view of a system 10 for accessing a targeted tissue region in the body for diagnostic and/or therapeutic purposes. FIG. 1 generally shows the system 10 deployed in the left atrium of the heart. Alternatively, system 10 can be deployed in other regions of the heart, such as the left ventricle, right atrium, or right ventricle. While the illustrated embodiment shows system 10 being used for ablating myocardial tissue, system 10 (and the methods described herein) may alternatively be configured for use in other tissue ablation applications, such as procedures for ablating tissue in the prostrate, brain, gall bladder, uterus, nerves, blood vessels and other regions of the body, including in systems that are not necessarily catheter-based.

System 10 includes a mapping catheter or probe 14 and an ablation catheter or probe 16. Each probe 14/16 may be separately introduced into the selected heart region 12 through a vein or artery (e.g., the femoral vein or artery) using a suitable percutaneous access technique. Alternatively, mapping probe 14 and ablation probe 16 can be assembled in an integrated structure for simultaneous introduction and deployment in the heart region 12.

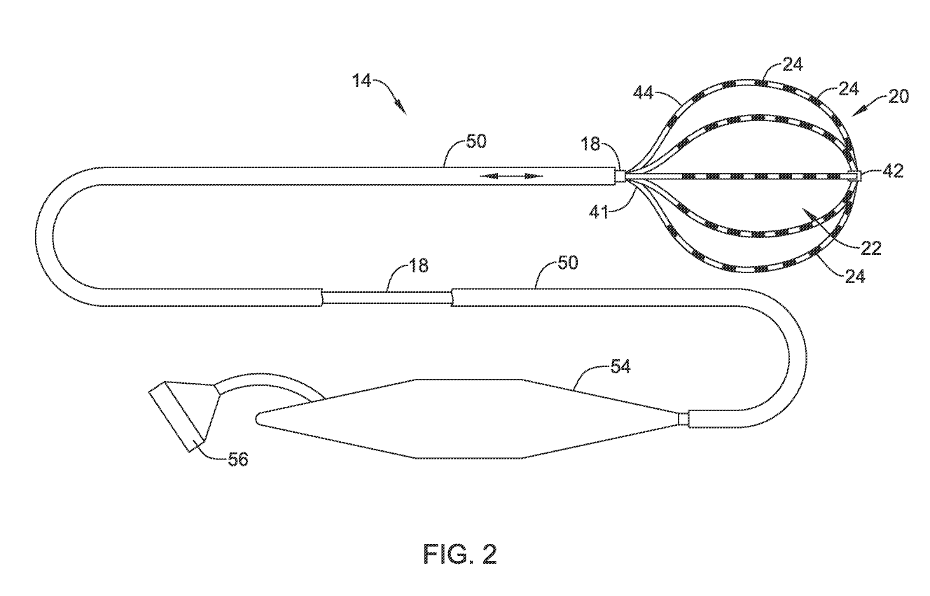

Mapping probe 14 may include flexible catheter body 18. The distal end of catheter body 18 carries three-dimensional multiple electrode structure 20. In the illustrated embodiment, structure 20 takes the form of a basket defining an open interior space 22 (see FIG. 2), although other multiple electrode structures could be used. Structure 20 carries a plurality of mapping electrodes 24 (not explicitly shown on FIG. 1, but shown on FIG. 2) each having an electrode location on structure 20 and a conductive member. Each electrode 24 may be configured to sense or detect intrinsic physiological activity, for example represented as electrical signals, in an anatomical region adjacent to each electrode 24.

In addition, electrodes 24 may be configured to detect activation signals of the intrinsic physiological activity within the anatomical structure. For example, intrinsic cardiac electrical activity may comprise repeating or semi-repeating waves of electrical activity with relatively large spikes in activity at the beginning of activation events. Electrodes 24 may sense such activation events and the times at which such activation events occur. Generally, electrodes 24 may sense activation events at different times as an electrical activity wave propagates through the heart. For instance, an electrical wave may begin near a first group of electrodes 24, which may sense an activation event at relatively the same time or within a relatively small window of time. As the electrical wave propagates through the heart, a second group of electrodes 24 may sense the activation event of the electrical wave at times later than the first group of electrodes 24.

Electrodes 24 are electrically coupled to processing system 32. A signal wire (not shown) may be electrically coupled to each electrode 24 on structure 20. The signal wires may extend through body 18 of probe 14 and electrically couple each electrode 24 to an input of processing system 32. Electrodes 24 sense cardiac electrical activity in the anatomical region, e.g., myocardial tissue, adjacent to their physical location within the heart. The sensed cardiac electrical activity (e.g., electrical signals generated by the heart which may include activation signals) may be processed by processing system 32 to assist a user, for example a physician, by generating processed output—e.g. an anatomical map (e.g., a vector field map, an activation time map) or a Hilbert transform diagram—to identify one or more sites within the heart appropriate for a diagnostic and/or treatment procedure, such as an ablation procedure. For example, processing system 32 may identify a near-field signal component (e.g., activation signals originating from cellular tissue adjacent to mapping electrodes 24) or an obstructive far-field signal component (e.g., activation signals originating from non-adjacent tissue). In such examples where structure 20 is disposed in an atrium of the heart, as in FIG. 1, the near-field signal component may include activation signals originating from atrial myocardial tissue whereas the far-field signal component may include activation signals originating from ventricular myocardial tissue. The near-field activation signal component may be further analyzed to find the presence of a pathology and to determine a location suitable for ablation for treatment of the pathology (e.g., ablation therapy).

Processing system 32 may include dedicated circuitry (e.g., discrete logic elements and one or more microcontrollers; application-specific integrated circuits (ASICs); or specially configured programmable devices, such as, for example, programmable logic devices (PLDs) or field programmable gate arrays (FPGAs)) for receiving and/or processing the acquired physiological activity. In some examples, processing system 32 includes a general purpose microprocessor and/or a specialized microprocessor (e.g., a digital signal processor, or DSP, which may be optimized for processing activation signals) that executes instructions to receive, analyze and display information associated with the received physiological activity. In such examples, processing system 32 can include program instructions, which when executed, perform part of the signal processing. Program instructions can include, for example, firmware, microcode or application code that is executed by microprocessors or microcontrollers. The above-mentioned implementations are merely exemplary, and the reader will appreciate that processing system 32 can take any suitable form for receiving electrical signals and processing the received electrical signals.

In addition, processing system 32 may be configured to measure the sensed cardiac electrical activity in the myocardial tissue adjacent to electrodes 24. For example, processing system 32 may be configured to detect cardiac electrical activity associated with a dominant rotor or divergent activation pattern in the anatomical feature being mapped. Dominant rotors and/or divergent activation patterns may have a role in the initiation and maintenance of atrial fibrillation, and ablation of the rotor path, rotor core, and/or divergent foci may be effective in terminating the atrial fibrillation. Processing system 32 processes the sensed cardiac electrical activity to generate a display of relevant characteristics. Such processed output may include isochronal maps, activation time maps, phase maps, action potential duration (APD) maps, Hilbert transform diagrams, vector field maps, contour maps, reliability maps, electrograms, cardiac action potentials and the like. The relevant characteristics may assist a user to identify a site suitable for ablation therapy.

Ablation probe 16 includes flexible catheter body 34 that carries one or more ablation electrodes 36. The one or more ablation electrodes 36 are electrically connected to radio frequency (RF) generator 37 that is configured to deliver ablation energy to the one or more ablation electrodes 36. Ablation probe 16 may be movable with respect to the anatomical feature to be treated, as well as structure 20. Ablation probe 16 may be positionable between or adjacent to electrodes 24 of structure 20 as the one or more ablation electrodes 36 are positioned with respect to the tissue to be treated.

Processing system 32 may output data to a suitable device, for example display device 40, which may display relevant information for a user. In some examples, device 40 is a CRT, LED, or other type of display, or a printer. Device 40 presents the relevant characteristics in a format useful to the user. In addition, processing system 32 may generate position-identifying output for display on device 40 that aids the user in guiding ablation electrode(s) 36 into contact with tissue at the site identified for ablation.

FIG. 2 illustrates mapping catheter 14 and shows electrodes 24 at the distal end suitable for use in system 10 shown in FIG. 1. Mapping catheter 14 may include flexible catheter body 18, the distal end of which may carry three-dimensional multiple electrode structure 20 with mapping electrodes or sensors 24. Mapping electrodes 24 may sense cardiac electrical activity, including activation signals, in the myocardial tissue. The sensed cardiac electrical activity may be processed by the processing system 32 to assist a user in identifying the site or sites having a heart rhythm disorder or other myocardial pathology via generated and displayed relevant characteristics. This information can then be used to determine an appropriate location for applying appropriate therapy, such as ablation, to the identified sites, and to navigate the one or more ablation electrodes 36 to the identified sites.

The illustrated three-dimensional multiple electrode structure 20 comprises base member 41 and end cap 42 between which flexible splines 44 generally extend in a circumferentially spaced relationship. As discussed herein, structure 20 may take the form of a basket defining an open interior space 22. In some examples, the splines 44 are made of a resilient inert material, such as Nitinol, other metals, silicone rubber, suitable polymers, or the like and are connected between base member 41 and end cap 42 in a resilient, pretensioned condition, to bend and conform to the tissue surface they contact. In the example illustrated in FIG. 2, eight splines 44 form three-dimensional multiple electrode structure 20. Additional or fewer splines 44 could be used in other examples. As illustrated, each spline 44 carries eight mapping electrodes 24. Additional or fewer mapping electrodes 24 could be disposed on each spline 44 in other examples of three-dimensional multiple electrode structure 20. In the example illustrated in FIG. 2, structure 20 is relatively small (e.g., 40 mm or less in diameter). In alternative examples, structure 20 is even smaller or larger (e.g., less than or greater than 40 mm in diameter).

Slidable sheath 50 may be movable along the major axis of catheter body 18. Moving sheath 50 distally relative to catheter body 18 may cause sheath 50 to move over structure 20, thereby collapsing structure 20 into a compact, low profile condition suitable for introduction into and/or removal from an interior space of an anatomical structure, such as, for example, the heart. In contrast, moving sheath 50 proximally relative to the catheter body may expose structure 20, allowing structure 20 to elastically expand and assume the pretensioned position illustrated in FIG. 2.

A signal wire (not shown) may be electrically coupled to each mapping electrode 24. The signal wires may extend through body 18 of mapping catheter 14 (or otherwise through and/or along body 18) into handle 54, in which they are coupled to external connector 56, which may be a multiple pin connector. Connector 56 electrically couples mapping electrodes 24 to processing system 32. It should be understood that these descriptions are just examples. Some addition details regarding these and other example mapping systems and methods for processing signals generated by a mapping catheter can be found in U.S. Pat. Nos. 6,070,094, 6,233,491, and 6,735,465, the disclosures of which are hereby expressly incorporated herein by reference.

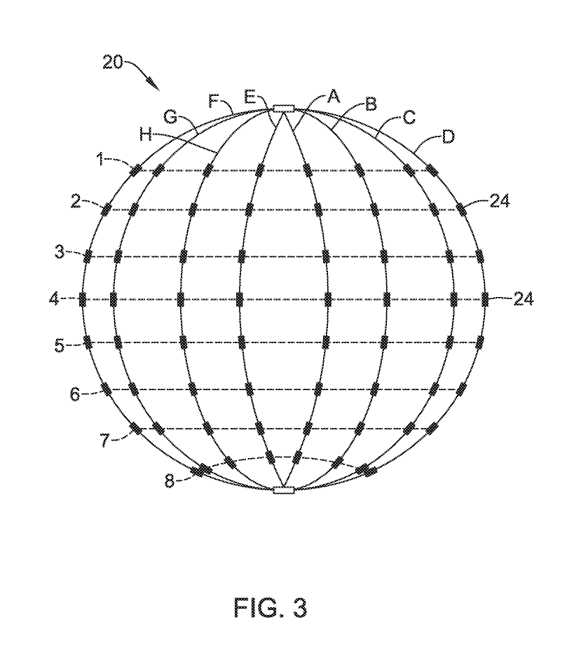

To illustrate the operation of system 10, FIG. 3 is a schematic side view of an example of basket structure 20 including a plurality of mapping electrodes 24. In the illustrated example, the basket structure includes 64 mapping electrodes 24. Mapping electrodes 24 are disposed in groups of eight electrodes (labeled 1, 2, 3, 4, 5, 6, 7, and 8) on each of eight splines (labeled A, B, C, D, E, F, G, and H). While an arrangement of sixty-four mapping electrodes 24 is shown disposed on basket structure 20, mapping electrodes 24 may alternatively be arranged in different numbers (more or fewer splines and/or electrodes), on different structures, and/or in different positions. In addition, multiple basket structures can be deployed in the same or different anatomical structures to simultaneously obtain signals from different anatomical structures.

After basket structure 20 is positioned adjacent to the anatomical structure to be treated (e.g. left atrium, left ventricle, right atrium, or right ventricle of the heart), processing system 32 may be configured to record the cardiac electrical activity from each electrode 24 channel. Further, the recorded cardiac electrical activity may be related to the physiological activity of the adjacent anatomical structure. For instance, cardiac electrical activity sensed by electrodes 24 may include activation signals which may indicate an onset of physiological activity (e.g. contraction of the heart). Further, cardiac electrical activity corresponding to physiological activity may be sensed in response to intrinsic physiological activity (e.g. intrinsically generated electrical signals) or based on a predetermined pacing protocol instituted by at least one of the plurality of electrodes 24 (e.g. delivered electrical signals delivered by a pacing device).

It should be noted that while much of the discussion herein relates to the use of system 10 within the heart, in some instances system 10 may be utilized in other areas of the body in addition to computed, simulated and/or theoretical computations. For example, embodiments may be applied to neurological activity, endocardial and/or epicardial activity, unipolar measurements, bipolar measurements, both unipolar and bipolar measurements, or the like. In other words, the disclosed embodiments and/or techniques may be applied to any electrical measurement and/or any electrical activity, real or computed.

The arrangement, size, spacing and location of electrodes along a constellation catheter or other mapping/sensing device, in combination with the specific geometry of the targeted anatomical structure, may contribute to the ability (or inability) of electrodes 24 to sense, measure, collect and transmit electrical activity of cellular tissue. As stated, because splines 44 of a mapping catheter, constellation catheter or other similar sensing device are bendable, they may conform to a specific anatomical region in a variety of shapes and/or configurations. Further, at any given position in the anatomical region, structure 20 may be manipulated such that one or more splines 44 may not contact adjacent cellular tissue. For example, splines 44 may twist, bend, or lie atop one another, thereby separating splines 44 from nearby cellular tissue. Additionally, because electrodes 24 are disposed on one or more of splines 44, they also may not maintain contact with adjacent cellular tissue. Electrodes 24 that do not maintain contact with cellular tissue may be incapable of sensing, detecting, measuring, collecting and/or transmitting electrical activity information. Further, because electrodes 24 may be incapable of sensing, detecting, measuring, collecting and/or transmitting electrical activity information, processing system 32 may be incapable of accurately displaying diagnostic information and/or processed output. For example, some necessary information may be missing and/or displayed inaccurately.

In addition to that stated above, electrodes 24 may not be in contact with adjacent cellular tissue for other reasons. For example, manipulation of mapping catheter 14 may result in movement of electrodes 24, thereby creating poor electrode-to-tissue contact. Further, electrodes 24 may be positioned adjacent fibrous, dead or functionally refractory tissue. Electrodes 24 positioned adjacent fibrous, dead or functionally refractory tissue may not be able to sense changes in electrical potential because fibrous, dead or functionally refractory tissue may be incapable of depolarizing and/or responding to changes in electrical potential. Finally, far-field ventricular events and electrical line noise may distort measurement of tissue activity.

However, electrodes 24 that contact healthy, responsive cellular tissue may sense a change in the voltage potential of a propagating cellular activation wavefront. The change in voltage potential of cellular tissue may be sensed, collected and displayed as an electrogram. An electrogram may be a visual representation of the change in voltage potential of the cellular tissue over time. Additionally, it may be desirable to define a specific characteristic of an electrogram as a “fiducial” point of the electrical signal. For purposes of this disclosure, a fiducial point may be understood as a characteristic of an electrogram that can be utilized as an identifying characteristic of cellular activation. Fiducial points may correspond to the peak magnitude, change in slope, and/or deflection of the electrical signal. It is contemplated that fiducial points may include other characteristics of an electrogram or other signal used to generate diagnostic and/or processed output. Further, fiducial points may be identified manually by a clinician and/or automatically by processing system 32.

An electrogram representing a change in voltage potential over time may be defined as visually displaying the electrical signal in the “time domain.” However, it is generally understood that any electrical signal has a corollary representation in the “frequency domain.” Transforms (e.g. Fourier, Fast Fourier, Wavelet, Wigner-Ville) may be utilized to transform signals between the time domain and frequency domain, as desired. Electrical signals also have a corollary representation in the analytic domain which can be obtained through transforms such as the Hilbert transform.

Further, in a normal functioning heart, electrical discharge of the myocardial cells may occur in a systematic, linear fashion. Therefore, detection of non-linear propagation of the cellular excitation wavefront may be indicative of cellular firing in an abnormal fashion. For example, cellular firing in a rotating pattern may indicate the presence of dominant rotors and/or divergent activation patterns. Further, because the presence of the abnormal cellular firing may occur over localized target tissue regions, it is possible that electrical activity may change form, strength or direction when propagating around, within, among or adjacent to diseased or abnormal cellular tissue. Identification of these localized areas of diseased or abnormal tissue may provide a user with a location for which to perform a therapeutic and/or diagnostic procedure. For example, identification of an area including reentrant or rotor currents may be indicative of an area of diseased or abnormal cellular tissue. The diseased or abnormal cellular tissue may be targeted for an ablative procedure. Various processed outputs, such as those described above, may be used to identify areas of circular, adherent, rotor or other abnormal cellular excitation wavefront propagation.

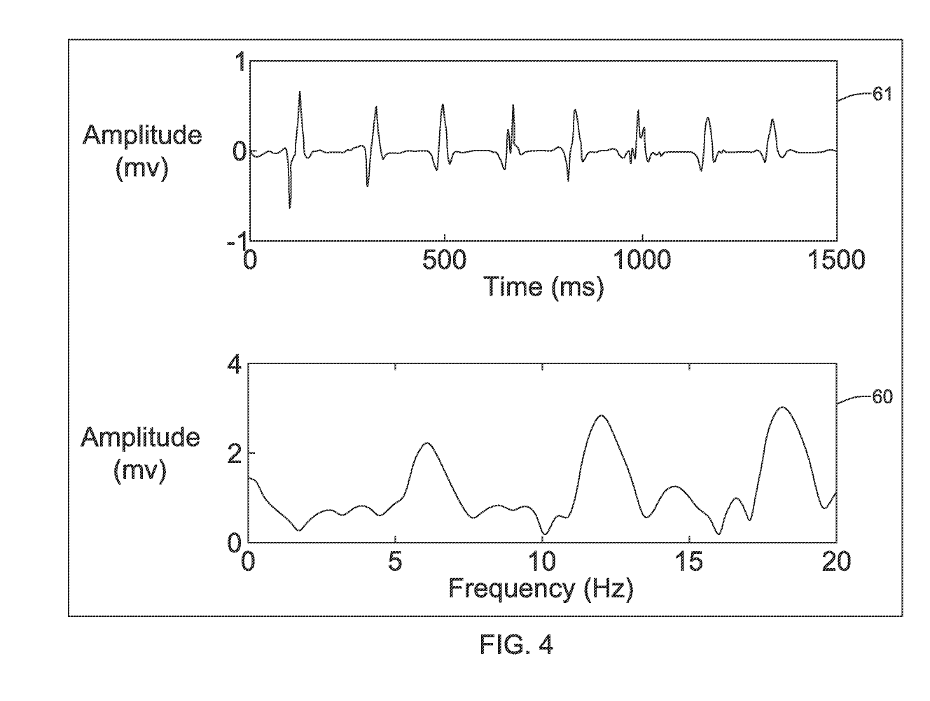

In at least some embodiments, the process of generating processed output may begin by collecting signals from one or more of sixty-four electrodes 24 on structure 20. As stated above, the sensed signals may be collected and displayed in the time domain. However, in at least one embodiment, signals displayed in the time domain may be transformed into the frequency domain to further generate processed output. As stated above, transforms such as the Fourier Transform, Fast Fourier Transform, or any other transform that produces frequency and power information for a signal may be utilized to transform signals between the time and frequency domains. FIG. 4 illustrates an example electrogram signal in the time domain 60 along with its corresponding frequency representation in the frequency domain 62.

Additionally, in some instances it may be desirable to analyze frequency representations over a time interval. For example, it may be desirable to analyze collected signal data as a time-frequency representation. In some instances, a time-frequency representation may be referred to as a spectrogram. For purposes of this disclosure, the terms time-frequency representation and spectrogram are used interchangeably.

A spectrogram may represent the magnitude of frequencies corresponding to cellular tissue response as it varies with time (or another variable). In some instances, transforms such as the Fourier Transform, Short-Time Fourier Transform, Wavelet (e.g. Morlet) transform, or any other transform that produces frequency and power information for a signal may be utilized to generate a spectrogram.

Additionally, in some instances it may be desirable to combine and/or utilize one or more transforms in conjunction to produce a spectrogram. For example, a Wavelet transform may be utilized in conjunction with the Fourier transform. Specifically, a continuous wavelet transform, such as a Morlet wavelet, may be used in conjunction with a Fourier transform. Utilizing particular combinations of wavelets and transforms (e.g. continuous wavelet transform and Fourier transform, etc.) may provide a more efficient method of creating a spectrogram as compared to conventional methodologies.

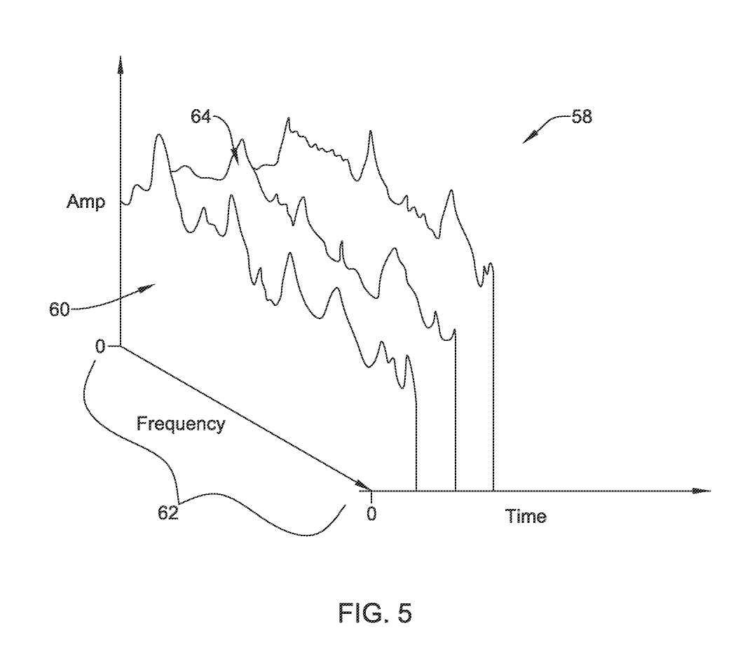

FIG. 5 shows a three-dimensional visual representation of example spectrogram 58. Spectrogram 58 may correspond to an example electrode 24 on multiple electrode structure 20. It should be understood that while spectrogram 58 is displayed visually in FIG. 5, processing system 32 may generate the data necessary to reconstruct a spectrogram without actually creating a visual display of the spectrogram. Further, processing system 32 may utilize the collected data independent of visually displaying a spectrogram.

As shown in FIG. 5, the spectrogram may display a frequency spectrum 60 which varies in magnitude over a range of frequencies 62. In practice, frequency range 62 may correspond to frequencies included in the original, collected electrical signals and/or a frequency range selected by a user. Additionally, processing system 32 may select a range of frequencies for which data is utilized from one or more of the signals collected from the sixty-four electrodes 24 on structure 20. For example, a frequency range of 3-7 Hz has been shown (empirically) to be a frequency range in which abnormal cardiac electrical activity occurs. For example, atrial fibrillation may occur predominantly in the frequency range of 3-7 Hz. It is contemplated that other abnormal atrial events may also occur within this frequency range. However, it should be understood that abnormal cardiac activity may occur in frequency ranges other than 3-7 Hz.

Additionally, it should be understood that the selected and/or filtered frequency range may be greater or less than 3-7 Hz (e.g. each limit could be modified by ±2-10 Hz). Selecting or ignoring data within a particular frequency range (e.g. in accordance with the range expected for a certain application) may improve the techniques and/or processed output of the embodiments disclosed herein. For example, the frequency range may be a narrower range (e.g. 3-7 Hz, 2-10 Hz, 5-20 Hz), or may be a larger range (e.g. 0-60 Hz, 5-100 Hz, 0-200 Hz).

As shown in FIG. 5, frequency spectrum 60 may correspond to a portion of the time interval over which original electrical signals were sensed and collected. Further, a frequency spectrum may change over the time interval. For example, a second frequency spectrum 64 may occur at a second time interval (as compared to the time interval corresponding to frequency spectrum 62). As shown, frequency spectrum 64 may be different from frequency spectrum 60. The difference between frequency spectrum 64 and frequency spectrum 60 may be due to a change in the magnitude of the spectrum with respect to frequency values over time. Further, the change in magnitude of the spectrum with respect to frequency values over time may correspond to a changing cellular tissue response underlying the original sensed and collected electrical signals.

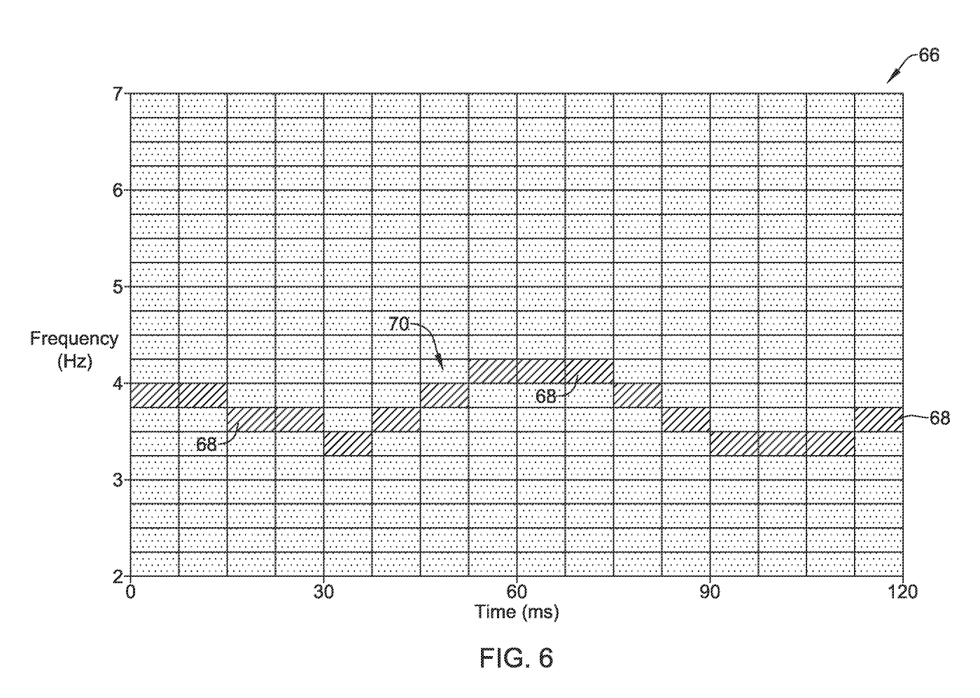

In addition to that displayed in FIG. 5, a two-dimensional spectrogram 66 is shown in FIG. 6. As shown in FIG. 6, spectrogram 66 may convey the same information as spectrogram 58. However, the information may be presented in a different format. For example, in FIG. 6, the time interval may be displayed on the X-axis, while the frequency range may be displayed on the Y-axis. Further, the magnitude values for each frequency may be conveyed visually. For example, the magnitude values may be conveyed by a color spectrum. In other words, a range of colors may indicate the relative magnitude of a given frequency. The example disclosed herein is merely illustrative—other methods for displaying the spectrogram (including frequency variability over time) and/or the magnitude of a given frequency are contemplated. For example, magnitude values may be indicated by texture.

In some instances, electrical signals sensed and collected by electrodes 24 may exhibit the same or very similar frequency characteristics over a given time interval. For example, electrical signals sensed and collected by electrodes 24 may exhibit the same or similar magnitudes at a given frequency over a given time interval. In other words, a given electrode may sense and collect electrical signals that exhibit a consistent magnitude at a given frequency over an interval of time. Further, similar frequency characteristics may be displayed, reproduced and/or identified on a spectrogram. For example, a spectrogram may convey magnitude values that are consistent at a given frequency over an interval of time.

Further, it is understood that the embodiments described above may be applicable to one or more of electrodes 24 on multiple electrode structure 20. For example, in some instances it may be desirable to generate a spectrogram for one or more of electrodes 24 on multiple electrode structure 20. Further, it may be desirable to apply one or more statistical analysis techniques across one or more spectrograms to better understand and/or display the underlying electrical information sensed by electrodes 24. In some instances, it may be desirable to apply statistical analysis techniques to a particular frequency characteristic across one or more electrodes 24 on multiple electrode structure 20.

It is contemplated that a variety of frequency characteristics may be used to compare the spectrograms of electrodes 24 on multiple electrode structure 20. For example, the specific frequency characteristic may be a frequency value having a maximum magnitude (herein called a “maximum frequency value”), a chirp, a sustained frequency value having a maximum magnitude (herein called a “sustained maximum frequency value”), a local frequency value having a maximum magnitude (herein called a “local maximum frequency value”) and/or other unique dominant frequency characteristics. These are just examples. Other characteristics are contemplated. In some instances a particular frequency characteristic may be referred to as a “mode.” In other instances, the mode may be referred to as a “dominant characteristic.” The dominant characteristic may occur at a frequency referred to as a “dominant frequency” and at a time point referred to as a “dominant time point.” Further, in some instances the mode may be referred to as a “dominant frequency value.” Additionally, it is contemplated that other user-defined dominant frequency values may be defined as modes. In some instances a single spectrogram for a single electrode may exhibit one or more modes identified by processing system 32.

As stated above, in some instances it may be desirable to apply statistical analysis techniques to a particular frequency characteristic for one or more electrodes 24 on multiple electrode structure 20. For example, processing system 32 may construct, determine or calculate a “composite” spectrogram common to one or more of the signals collected from each of the sixty-four electrodes 24 on structure 20. The composite spectrogram may be constructed, determined or calculated by performing one or more mathematical, statistical or computational operations involving one or more of the signals collected from the sixty-four electrodes 24 on structure 20. FIG. 6 illustrates an example composite spectrogram 66. FIG. 6 illustrates composite spectrogram 66 generated by calculating the median amplitude and/or power value (e.g. statistical median) for each frequency and time point across multiple (e.g. all) sixty-four spectrograms corresponding to the sixty-four electrodes 24 on structure 20. As stated above, spectrogram 66 is a two-dimensional spectrogram where the magnitude values may be conveyed by a color spectrum, texture, pattern or the like. In other words, a range of colors/patterns may indicate the relative magnitude at a given frequency and time point. Calculating the statistical median for all frequencies and time points across one or more spectrograms is one of numerous possible methodologies processing system 32 may utilize to construct, determine or calculate a composite spectrogram. For example, processing system 32 may utilize the mean, median, mode or any other mathematical, statistical or computational operation to construct, determine or calculate a composite spectrogram.

Additionally, in some instances processing system 32 may determine, seek and/or track a particular frequency characteristic and/or mode of composite spectrogram 66. For example, processing system 32 may determine the maximum power values 68 (shown in FIG. 6 in cross hatching) for each frequency and time point across composite spectrogram 66. Further, the frequency and time point at which at which a particular maximum power value occurs may represent a “dominant frequency” and “dominant time point.” In some instances, the series and/or collection of modes (e.g. maximum frequency values 68) spanning the time period of composite spectrogram 66 may be referred to as a “fundamental frequency track” or “dominant frequency track.” FIG. 6, for example, illustrates fundamental frequency track 70 (including the collection and/or series of maximum power values 68) spanning a 120 ms time interval. In a two-dimensional spectrogram, fundamental frequency track 70 may appear as a line spanning across composite spectrogram 66.

As stated above, processing system 32 may utilize any mathematical, statistical or computational operation (e.g. mean, median, mode, etc.) to construct, determine or calculate a composite spectrogram. Additionally, the frequency values may be derived from a variety of computational operations. Further, it should be understood that processing system 32 may not have to calculate a composite spectrogram in order to generate, determine, select or derive a frequency characteristics, fundamental frequencies, fundamental frequency tracks or the like. Rather, it may be possible for processing system 32 to determine unique spectrogram characteristics by analyzing the data from one or more of the signals collected from the sixty-four electrodes 24 on structure 20 independently of determining a composite spectrogram.

In some instances, it may be desirable to generate a filter corresponding to a composite spectrogram. For example, filtering a composite spectrogram may be accomplished by utilizing a filtering process and/or methodology that selects desirable frequency values and excludes other, less-desirable, frequency values. It should be appreciated that in certain signal processing techniques, particularly techniques incorporating wavelet transforms, units of frequency may have a corresponding unit referred to as “scales.” Therefore, embodiments disclosed herein are understood to reference “frequency” (and units thereof) interchangeably with “scales.”

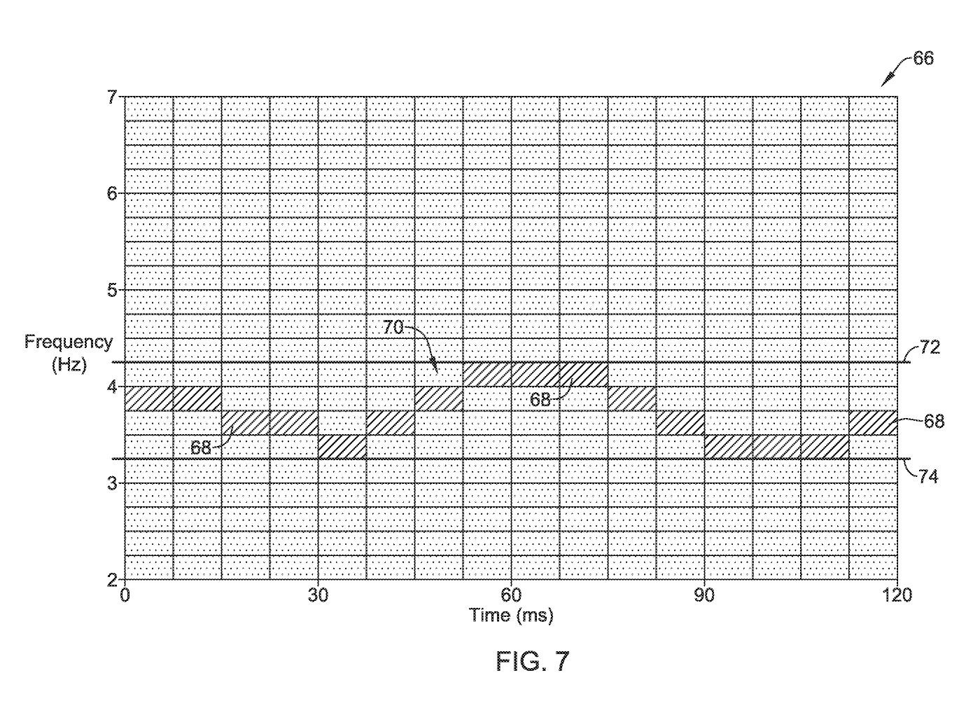

FIG. 7 illustrates a filtering methodology to filter desired frequency values from example composite spectrogram 66. In this example filtering methodology, processing system 32 may filter desirable frequency values based on a frequency range corresponding to the maximum and minimum frequencies of fundamental frequency track 70. For example, the filtering methodology may “select” all frequency values having a frequency between the maximum and minimum frequency bounds “encompassing” fundamental frequency track 70. In FIG. 7, the maximum frequency bound (depicted by bold line 72) is approximately equal to 4.25 Hz. Similarly, the minimum frequency bound (depicted by bold line 74) is approximately equal to 3.25 Hz. Therefore, processing system may filter composite spectrogram 66 by “selecting” all frequency values between 3.25 and 4.25 Hz. All remaining frequency values may be filtered out (e.g. ignored). In some instances, the filtering process depicted in Fig. may be referred to as a “rectangular” band filtering process and/or methodology.

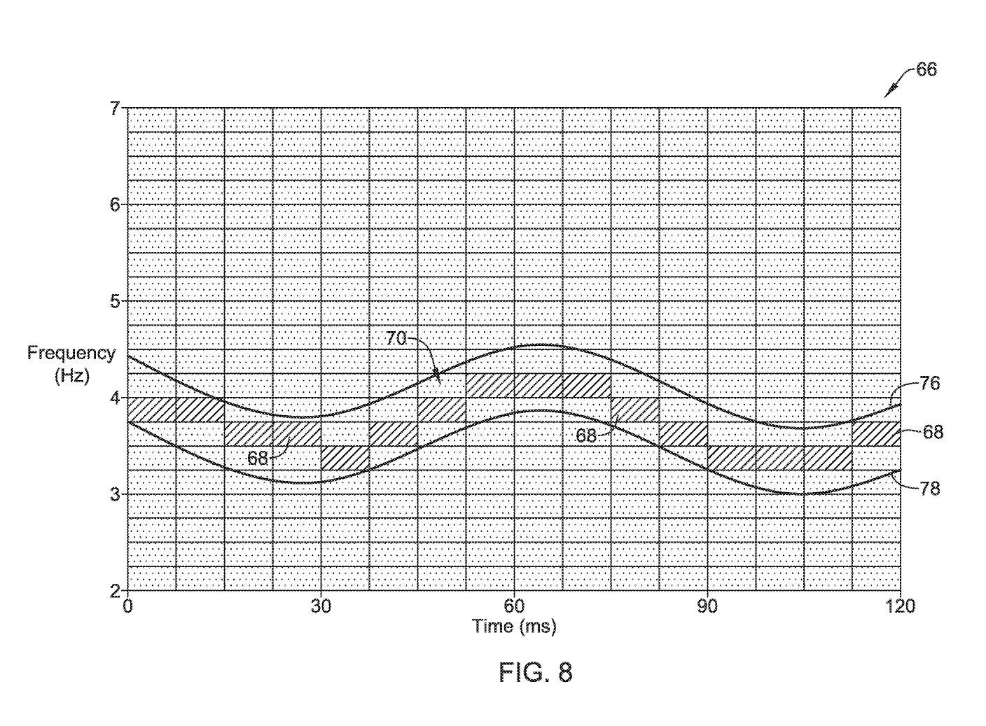

FIG. 8 illustrates another example filtering methodology to filter desired frequency values from the example composite spectrogram 66. In this example, processing system 32 may filter desirable frequency values based on a frequency range corresponding to a maximum and minimum frequency bound calculated from individual frequency values included in fundamental frequency track 70. For example, the maximum and minimum frequency bounds (as described with respect to FIG. 7), may be calculated as a percentage of the frequency corresponding to each frequency value of fundamental frequency track 70. For example, using 10% as an example percent offset, the maximum and minimum frequency bounds for a frequency value occurring at 4 Hz would be 3.6 Hz to 4.4 Hz. It can be appreciated that the maximum and minimum frequency bounds illustrated in FIG. 8 may result if the same percent offset was applied uniformly to each frequency corresponding to each frequency value included in fundamental frequency band 70. In FIG. 8, the maximum bound is depicted by bold line 76, while the minimum bound is depicted by minimum bound 78. In some instances, the filtering process depicted in FIG. 8 may be referred to as an “envelope” band filtering process and/or methodology.

With respect to the envelope band filtering process, a 10% offset is merely illustrative. Other offset percentages, offset algorithms and/or offset methodologies are appreciated. For example, a fixed frequency value or scale may be applied to each frequency corresponding to the frequency values of a fundamental frequency track. Additionally, similar to the methodology described with respect to FIG. 7, the filtering methodology of FIG. 8 may “select” all frequency values having a frequency between the maximum and minimum frequency bounds “encompassing” fundamental frequency track 70.

In addition to utilizing a filtering process and/or methodology to filter desired frequency values from undesired frequency values of composite spectrogram 66, processing system 32 may generate a binary mask. A binary mask may include a collection of binary input, values and/or representations (e.g. “0” or “1”) corresponding to the filtered composite spectrograms described above with respect to FIGS. 7 & 8. In some instances, generating the binary mask includes a dominant frequency region defined between a maximum frequency and a minimum frequency. In other instances, generating the binary mask may include a dominant frequency region defined between a first bound that corresponds to a percentage increase for each dominant frequency value over time and a second bound that corresponds to a percentage decrease for each dominant frequency value over time.

For example, included frequency values (e.g. desirable frequency values within the minimum and maximum frequency bounds) may be assigned a binary value of 1. Correspondingly, frequency values excluded by the filtering methodology may be assigned a binary value of 0.

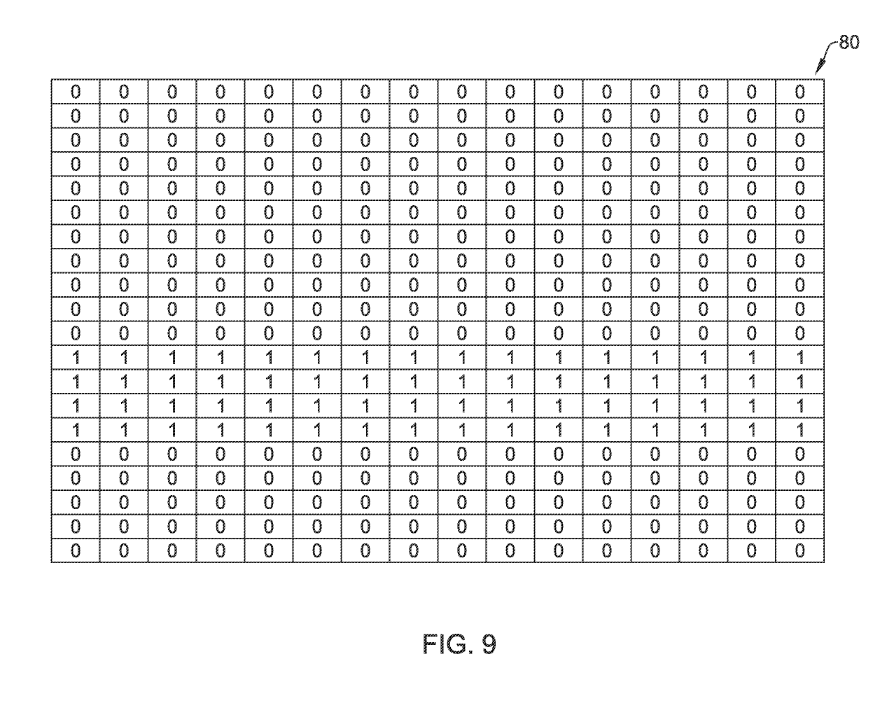

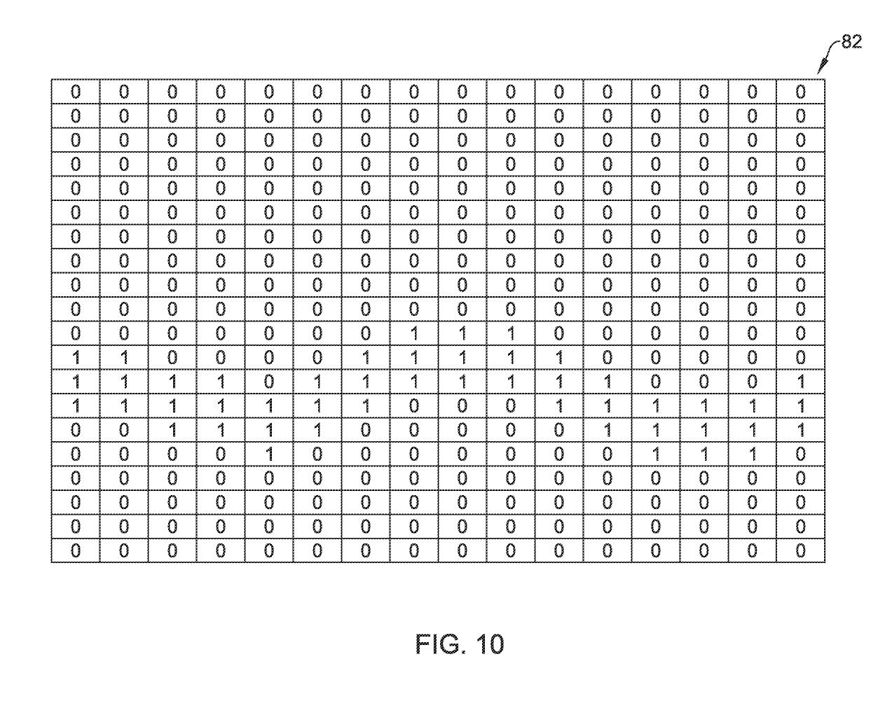

FIGS. 9 & 10 are illustrated depictions of binary masks 80/82 corresponding the filtered composite spectrums described in FIGS. 7 & 8, respectively. As illustrated in FIGS. 9&10, the frequency values within the maximum and minimum frequency bounds have been assigned an integer value of “1,” while frequency values outside the maximum and minimum frequency bounds have been assigned a frequency value of “0.” In some instances, the binary mask 80 depicted in FIG. 9 may be referred to as a “rectangular binary mask,” while binary mask 82 depicted in FIG. 10 may be referred to as an “envelope binary mask.”

It is understood that while the above discussion contemplates generating a time-frequency mask (e.g. binary mask disclosed herein) and applying that mask to a given time-frequency distribution (e.g. spectrograms derived from sensed signals), the same process may be applied to equivalent representations in the time domain. For example, a time-frequency mask may have an equivalent representation as a time-varying time-domain filter. Likewise, a time-frequency distribution (e.g. spectrogram) may have an equivalent time-domain representation. Therefore, similarly to that disclosed herein with respect to applying a time-frequency mask to time-frequency representation, a time-varying time-domain filter may be applied to a time-domain representation.

In addition to any of the embodiments described herein, to better understand and/or display the underlying electrical information sensed by electrodes 24 it may be desirable to simplify and/or filter one or more of the sixty-four spectrograms (or the spectrogram's equivalent time-domain representation) generated from the original, sensed signals corresponding to electrodes 24. The filtered spectrograms may be referred to as “alternate spectrograms.” In some instances, alternate spectrograms for all sixty-four electrodes 24 may be generated by multiplying each original spectrogram by a binary mask generated from a composite spectrogram (as described above with respect to FIGS. 6-10).

Additionally, it may be desirable for processing system 32 to create a diagnostic display corresponding to the individual alternate spectrograms (and by extension, all sixty-four electrodes 24). In some instances, a diagnostic display may be generated by displaying spectral-temporal patterns and/or phase values corresponding to each alternate spectrogram. In some instances the spectral-temporal patterns may include sinusoid signals and/or patterns displaying time-varying frequency. Further, the frequency of the sinusoid signal and/or pattern may vary within the range of dominant frequencies or scales that were filtered from the original spectrogram (e.g. between the minimum and maximum frequency bounds disclosed herein). The time variation in frequencies may be indicative of the potential interplay of multiple and closely spaced modes. Some additional details regarding multiple and closely spaced modes can be found in U.S. patent application titled “Medical System for Mapping Cardiac Tissue” (Atty. Docket No. 1001.3620100) and U.S. patent application titled “Medical System for Mapping Cardiac Tissue” (Atty. Docket No. 1001.3621100) the disclosures of which are hereby expressly incorporated herein by reference.

As stated, processing system 32 may determine the sinusoidal pattern representation and/or phase value correlated to the alternate spectrograms collected from electrodes 24 on structure 20. For example, an Inverse Continuous Wavelet transform may be applied to each individual alternate spectrogram to generate a sinusoidal pattern and/or phase value for each electrode 24. Further, each derived sinusoidal pattern with a corresponding phase offset may be utilized to create a dynamic “movie” or “dynamic map” corresponding to the particular electrode from which the alternate spectrogram was derived. A movie or dynamic map may provide a medium that allows better visualization of wavefront propagation and/or the focal impulse of a particular pathology via a summary characteristic (e.g. activation time, phase, etc.). In some embodiments, the visual display (e.g. movie, dynamic map, phase map etc.) may be portrayed on an anatomical representation of a cardiac chamber of interest. Additionally, the visual display (e.g. movie, dynamic map, phase map, etc.) may correspond to the first and/or second dominant frequency values changing over multiple heart beasts and/or over various cardiac regions or chambers. Some additional details regarding creating a dynamic phase map from sinusoid representations can be found in U.S. patent application titled “Medical Devices for Mapping Cardiac Tissue” (Atty. Docket No. 1001.3562100) the disclosure of which is hereby expressly incorporated herein by reference.

It should be understood that processing system 32 may selectively eliminate some of the collected signals before performing the techniques and/or embodiments disclosed herein. For example, it may be beneficial to eliminate signals collected by electrodes that are not in electrical contact, or in poor electrical contact, with excitable cellular tissue of the heart. Such signals may not provide useful information and can skew results of the above described techniques.

Alternatively, instead of eliminating collected signals that are not providing useful information, processing system 32 may instead interpolate or estimate the value of any signal which is not otherwise providing desirable information. Processing system 32 may utilize the interpolated or estimated data (e.g. signal data) to better calculate, determine or generate useful processed data and/or smooth, refine, or present processed data in a more desirable manner.

It is contemplated that any of the disclosed methods may be implemented across multiple beats, excitations or cardiac pacing time intervals. Further, data collected over multiple heart beats and/or excitations may be analyzed using statistical methodologies and applied to the disclosed methods. For example, activation times may be collected over a series of heart beats and/or pulses. A statistical distribution of the collected activation times may be calculated, analyzed and incorporated into disclosed methods.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system for mapping the electrical activity of the heart, the system comprising:
a processor, wherein the processor is capable of:
sensing a plurality of signals with a plurality of electrodes positioned within the heart;
collecting a plurality of signals corresponding to the plurality of electrodes, wherein collecting the plurality of signals occurs over a time period;
generating a plurality of time-frequency distributions corresponding the plurality of signals;
generating a composite time-frequency distribution corresponding to the plurality of signals;
generating a filter from the composite time-frequency distribution; and
applying the filter to the plurality of signals or to the plurality of time-frequency distributions.

2. The system of claim 1, wherein generating a plurality of time-frequency distributions utilizes at least one Fourier transform, Short-Time Fourier transform and/or a Wavelet transform.

3. The system of claim 2, wherein generating a plurality of time-frequency distributions further comprises utilizing a Continuous Wavelet Transform in conjunction with a Fourier transform.

4. The system of claim 2, wherein each of the plurality of time-frequency distributions includes one or more frequency values occurring at one or more frequencies and one or more time points, and wherein generating a composite time-frequency distribution includes determining the mode, median or mean of all the time-frequency distributions at each frequency and time point.

5. The system of claim 4, wherein generating a filter from the composite time-frequency distribution includes identifying a dominant frequency value for each time point of the composite time-frequency distribution, and wherein each dominant frequency value corresponds to a dominant frequency characteristic.

6. The system of claim 5, wherein the dominant frequency characteristic includes a maximum frequency value, a chirp, a sustained maximum frequency value, a local maximum frequency and/or a unique dominant frequency characteristic.

7. The system of claim 5, wherein generating a filter from the composite time-frequency distribution further comprises generating a binary mask.

8. The system of claim 7, wherein generating the binary mask includes a dominant frequency region defined between a maximum frequency and a minimum frequency.

9. The system of claim 7, wherein generating the binary mask includes a dominant frequency region defined between a first bound that corresponds to a percentage increase for each dominant frequency value over time and a second bound that corresponds to a percentage decrease for each dominant frequency value over time.

10. The system of claim 7, wherein generating the filter from the composite time-frequency distribution further comprises multiplying the binary mask with each of the plurality of time-frequency distributions.

11. The system of claim 10, wherein multiplying the binary mask with each of the plurality of time-frequency distributions generates an alternate time-frequency distribution corresponding to each of the time-frequency distributions.

12. The system of claim 11, further comprising generating a visual display, and wherein the visual display includes displaying at least one visual indicator, and wherein the visual indicator corresponds to each alternate time-frequency distribution.

13. The system of claim 12, wherein generating a visual display includes displaying at least one spectral-temporal pattern corresponding to each of the alternate time-frequency distributions.

14. The system of claim 13, wherein displaying at least one spectral-temporal pattern includes performing an Inverse Continuous Waveform transform on each alternate time-frequency distribution.

15. The system of claim 12, wherein the visual display includes displaying a phase map and wherein the visual indicator is a color, texture or both.

16. A system for mapping the electrical activity of the heart, the system comprising:
a catheter shaft;
a plurality of electrodes coupled to the catheter shaft; and
a processor, wherein the processor is capable of:
sensing a plurality of signals with a plurality of electrodes positioned within the heart;
collecting a plurality of signals corresponding to the plurality of electrodes, wherein collecting the plurality of signals occurs over a time period, and wherein the time period includes one or more time points;
generating a plurality of time-frequency distributions corresponding to the plurality of signals;
generating a composite time-frequency distribution corresponding to the plurality of time-frequency distributions, wherein the composite time-frequency distribution includes one or more fundamental frequency values at each time point of the time period;
generating a filter from the composite time-frequency distribution; and
applying the filter to the plurality of time-frequency distributions.

17. The system of claim 16, wherein generating a filter from the composite time-frequency distribution further comprises generating a binary mask corresponding to the fundamental frequency values.

18. The system of claim 17, wherein applying the filter to the plurality of time-frequency distributions further comprises multiplying the binary mask with the time-frequency distributions to generate an alternate time-frequency distribution for each electrode.

19. The system of claim 18, further comprising creating visual display, wherein creating a visual display includes displaying a sinusoid corresponding to the alternate time-frequency distribution for each electrode.

20. A method for mapping the electrical activity of the heart, the method comprising:
positioning a mapping device in the heart, wherein the mapping device is coupled to a processor and wherein the processor is capable of:
sensing a plurality of signals with a plurality of electrodes positioned within the heart;
collecting a plurality of signals corresponding to the plurality of electrodes, wherein collecting the plurality of signals occurs over a time period;
generating a plurality of time-frequency distributions corresponding the plurality of signals;
generating a composite time-frequency distribution corresponding to the plurality of signals;
generating a filter from the composite time-frequency distribution; and
applying the filter to the plurality of time-frequency distribution.
